(19) United States
(12) Patent Application Publication
(10) Pub. No.: US 2023/0144382 A1
(43) Pub. Date: May 11, 2023

(54) Temperature Measurement Device and Temperature Measurement Method

(71) Applicant: Nippon Telegraph and Telephone Corporation, Tokyo (JP)

(72) Inventors: Daichi Matsunaga, Tokyo (JP); Yujiro Tanaka, Tokyo (JP); Michiko Seyama, Tokyo (JP)

(21) Appl. No.: 17/917,604

(22) PCT Filed: Apr. 28, 2020
(86) PCT No.: PCT/JP2020/018096

§ 371 Date: Oct. 7, 2022

(51) Int. Cl. A61B 5/0205; A61B 5/026

(52) U.S. Cl. CPC A61B 5/02055; A61B 5/026; A61B 2562/0271; A61B 2560/0238

(57) ABSTRACT

An embodiment a temperature measuring device including a blood flow meter configured to measure a first blood flow near a skin surface of a subject, a sensor configured to measure a first temperature and a first heat flux of the skin surface of the subject, a storage device configured to store a second blood flow of the subject, the second blood flow being measured before the first blood flow, a thermal resistance deriver configured to derive a first thermal resistance of the subject based on an amount of change between the first and second blood flows, and a temperature calculator configured to calculate an internal temperature of the subject based on the first temperature, the first heat flux, and the first thermal resistance.

### CROSS-REFERENCE TO RELATED APPLICATIONS

This application is a national phase entry of PCT Application No. PCT/JP2020/018096, filed on Apr. 28, 2020, which application is hereby incorporated herein by reference.

### TECHNICAL FIELD

The present invention relates to a temperature measuring device and a temperature measuring method that measure the internal temperature of a subject such as a living body.

### BACKGROUND

In a substance, for example, a living body, when a certain depth is exceeded from the epidermis toward a deep body, there is a temperature area that is not affected by changes in outside air temperature, or the like, and the temperature of this area is called a deep body temperature or a core body temperature. On the other hand, the temperature of a surface layer of a living body that is susceptible to changes in outside air temperature is called a body surface temperature. The body surface temperature may be measured by a percutaneous thermometer in the related art. The body temperature measured by such a percutaneous thermometer in the related art may not reflect the deep body temperature. Therefore, it is difficult to directly measure the deep body temperature, which is the temperature in the deep area of the living body, like the body surface temperature.

Therefore, the inventor proposed a noninvasive deep body temperature measurement technology for measuring a skin surface heat flux H_{Skin} and a skin surface temperature T_{Skin} by a sensor installed on a skin surface and estimating a deep body temperature T_{Core} by using these measured values and biothermal resistance R_{Body} given by initial calibration (see NPL 1 and NPL 2). An Equation for estimating the deep body temperature T_{Core} is as follows.

T_{Core}=T_{Skin}+R_{Body}H_{Skin} (1)

The biothermal resistance R_{Body} is modeled as a constant because it is determined by the thickness from the skin surface to a deep body temperature area at a sensor installation site. However, when the blood flow in capillaries or arteriovenous anastomosis changes due to a warm bath, exercise, or the like, the actual thermal resistance of a living body may change from a value given by initial calibration, resulting in a problem in that an error occurs in an estimated value of the deep body temperature T_{Core}.

### CITATION LIST

### Non Patent Literature

NPL 1: Daichi Matsunaga, Yujiro Tanaka, Tomoko Seyama, “Study for miniaturization of a noninvasive deep body temperature sensor considering convection change”, 2020 Institute of Electronics, Information and Communication Engineers (IEICE) General Conference, Communication lecture Proceedings 1, B-19-9, 2020
NPL 2: Daichi Matsunaga, Yujiro Tanaka, Tomoko Seyama, “Study of a noninvasive deep body temperature estimation method for convection change in outside air”, 2019 Institute of Electronics, Information and Communication Engineers (IEICE) Communication Society Conference, Communication lecture Proceedings 1, B-19-15, 2019

### SUMMARY

### Technical Problem

The present invention has been made to solve the above problems, and an object of the present invention is to provide a temperature measuring device and a temperature measuring method, capable of reducing an error in an estimated value of the internal temperature of a subject caused by a change in blood flow.

### Means for Solving the Problem

A temperature measuring device according to embodiments of the present invention includes: a blood flow meter configured to measure a blood flow near a skin surface of a subject; a sensor configured to measure a temperature and a heat flux of the skin surface of the subject; a storage unit configured to store an initial value of the blood flow in advance; a thermal resistance derivation unit configured to derive thermal resistance of the subject on the basis of an amount of change with respect to the initial value of the blood flow measured by the blood flow meter; and a temperature calculation unit configured to calculate an internal temperature of the subject on the basis of the temperature, the heat flux, and the thermal resistance.

Furthermore, a temperature measuring method according to embodiments of the present invention includes: a first step of measuring a blood flow near a skin surface of a subject; a second step of deriving thermal resistance of the subject on the basis of an amount of change with respect to an initial value of the blood flow stored in advance; a third step of measuring a temperature and a heat flux of the skin surface of the subject; and a fourth step of calculating an internal temperature of the subject on the basis of a measurement result in the third step and the thermal resistance derived in the second step.

### Effects of the Invention

According to embodiments of the present invention, deriving the thermal resistance of a subject on the basis of the amount of change in blood flow measured by a blood flow meter enables to reduce an error in an estimated value of the internal temperature of a subject caused by a change in blood flow.

### BRIEF DESCRIPTION OF THE DRAWINGS

FIG. 1 is a block diagram illustrating the configuration of a temperature measuring device according to an embodiment of the present invention.

FIG. 2 is a diagram illustrating a thermal equivalent circuit model of a sensor and a living body according to the embodiment of the present invention.

FIG. 3 is a flowchart for explaining operations of the temperature measuring device according to the embodiment of the present invention.

FIG. 4 is a diagram for explaining operations of the temperature measuring device according to the embodiment of the present invention.

FIG. 5 is a block diagram illustrating an example of the configuration of a computer that implements the temperature measuring device according to the embodiment of the present invention.

### DETAILED DESCRIPTION OF ILLUSTRATIVE EMBODIMENTS

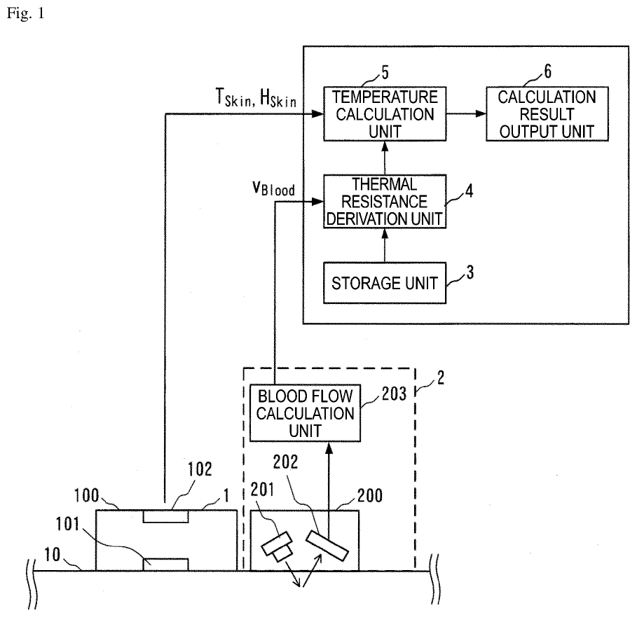

Hereinafter, an embodiment of the present invention will be described with reference to the drawings. FIG. 1 is a block diagram illustrating the configuration of a temperature measuring device according to an embodiment of the present invention. The temperature measuring device includes a sensor 1 that measures a temperature T_{Skin} of a skin surface of a living body 10 (subject) and a heat flux H_{Skin} on the skin surface, a laser Doppler blood flow meter 2 that measures a blood flow v_{Blood} near the skin surface of the living body 10, a storage unit 3 that stores an initial value v_{Blood} (o) of the blood flow v_{Blood} in advance, a thermal resistance derivation unit 4 that derives thermal resistance R_{Combined} of the living body 10 on the basis of the amount Δv_{Blood} of change with respect to the initial value v_{Blood} (o) of the blood flow v_{Blood}, a temperature calculation unit 5 that calculates a deep body temperature T (internal temperature) of the living body 10 on the basis of the temperature T_{Skin}, the heat flux H_{Skin}, and the thermal resistance R_{Combined}, and a calculation result output unit 6 that outputs the calculation result of the deep body temperature T_{Core}.

The sensor 1 includes a thermal insulation member wo, a temperature sensor 101 disposed on a surface of the thermal insulation member wo in contact with the skin of the living body 10, and a temperature sensor 102 disposed on a surface of the thermal insulation member wo on a side opposite to the surface in contact with the skin. By the temperature sensor 101, it is possible to measure the temperature T_{Skin} of the skin surface of the living body 10. Furthermore, it is possible to derive the heat flux H_{Skin} of the skin surface on the basis of a difference between the temperature T_{Skin} of the skin surface and a temperature T_{Upper} measured by the temperature sensor 102. The sensor 1 is attached to the skin surface of the living body 10 by, for example, a thermally conductive double-sided tape. The configuration illustrated in FIG. 1 is an example, and the sensor 1 may have a configuration different from that illustrated in FIG. 1.

The laser Doppler blood flow meter 2 includes a sensor probe 200 and a blood flow calculation unit 203. The sensor probe 200 is provided with a semiconductor laser 201 that irradiates the living body 10 with a laser beam and a photodiode 202 that receives reflected light from the living body 10. The blood flow calculation unit 203 calculates the blood flow v_{Blood} of the living body 10 on the basis of an electric signal output from the photodiode 202. Since the laser Doppler blood flow meter 2 is a well-known technology, detailed description thereof will be omitted.

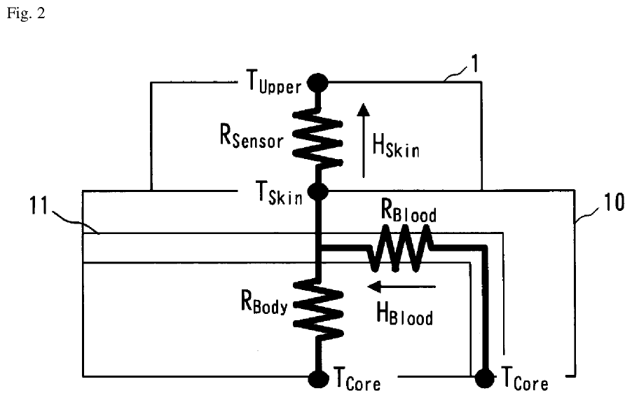

FIG. 2 is a diagram illustrating a thermal equivalent circuit model of the sensor 1 and the living body 10. In the present invention, not only the thermal resistance R_{Body} of the living body 10 is modeled, but also thermal energy transferred by blood flow is modeled. In FIG. 2, 11 denotes a blood vessel, T_{Upper} denotes the temperature of an upper surface of the sensor 1 on a side opposite to the surface in contact with the skin of the living body 10, R_{Sensor} denotes the thermal resistance of the sensor 1, R_{Blood} denotes heat resistance due to the blood flow, and H_{Blood} denotes a heat flux due to the blood flow.

Thermal resistance given by initial calibration is the combined resistance R_{Combined} of the thermal resistance R_{Body} of tissues (skin, fat, muscle, nerve, internal organs, bone, etc.), other than the blood of the living body 10, and the thermal resistance R_{Blood} due to the blood flow of the living body 10.

T_{Core}=T_{Skin}+R_{Combined}H_{Skin} (2)

In the initial calibration, when the initial value of the deep body temperature T_{core} of the living body 10, whose deep body temperature T_{Core} is to be measured, at a part around the sensor 1 is measured by, for example, a heat flow compensation method or an eardrum thermometer and at the same time, the skin surface temperature T_{Skin} and the skin surface heat flux H_{Skin} are measured by the sensor 1, the initial value R_{Combined} (o) of the combined resistance R_{Combined} can be obtained by Equation 2 above.

The thermal resistance R_{Body} is a constant, and the thermal resistance R_{Blood} is expressed as a function of the blood flow v_{Blood}. Consequently, the combined resistance R_{Combined} is a function of blood flow v_{Blood} as expressed by the equation below.

$$\begin{matrix}[ Math . 1 ] & \\ {R}_{Combined} = \frac{{R}_{Body} {R}_{Blood}}{{R}_{Body} + {R}_{Blood}} = f ( {v}_{B l o o d} ) & ( 3 )\end{matrix}$$

Therefore, a conversion table for the amount of change in the combined resistance R_{Combined} and the blood flow v_{Blood} is prepared in advance, and the initial value R_{Combined} (o) of the combined resistance R_{Combined} is updated from the amount of change in the blood flow v_{Blood} measured by a laser Doppler blood flow meter or the like, as expressed by the equation below.

$$\begin{matrix}[ Math . 2 ] & \\ {R}_{Combined} = {R}_{Combined} ( 0 ) f ( \frac{{v}_{Blood}}{{v}_{Blood} ( 0 )} ) & ( 4 )\end{matrix}$$

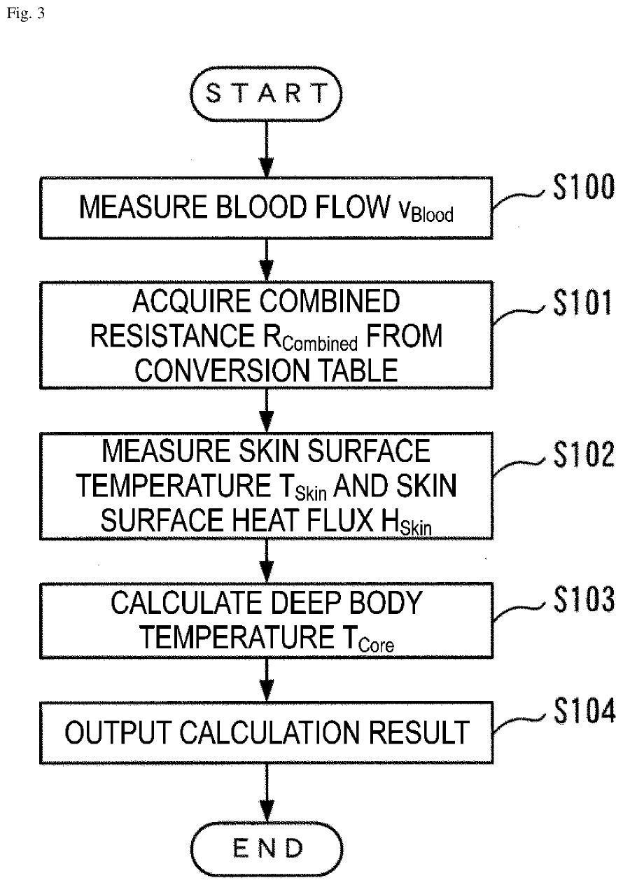

In this way, in the present embodiment, it is possible to reduce an error in the estimated value of the deep body temperature T_{Core} that occurs when the blood flow changes. FIG. 3 is a flowchart for explaining operations of the temperature measuring device of the present embodiment. The storage unit 3 of the temperature measuring device stores in advance a conversion table, in which the combined resistance R_{Combined} of the living body 10 is registered for each amount Δv_{Blood} of change in the blood flow v_{Blood}, and the initial value v_{Blood} (o) of the blood flow v_{Blood} measured by the laser Doppler blood flow meter 2 at the time of the initial calibration.

In order to generate the conversion table, the deep body temperature T_{Core} of the living body 10, whose deep body temperature T_{Core}, is to be measured, at the part around the sensor 1 is measured by, for example, a heat flow compensation method or an eardrum thermometer while monitoring the blood flow v_{Blood} at the part around the sensor 1 by the laser Doppler blood flow meter 2, and the skin surface temperature T_{Skin} and the skin surface heat flux H_{Skin} are measured by the sensor 1. Then, in a case where there is a change in the blood flow v_{Blood}, when the combined resistance R_{Combined} is calculated by Equation 2 above from the deep body temperature T_{Core}, the skin surface temperature T_{Skin}, and the skin surface heat flux H_{Skin} when the blood flow v_{Blood} changes from a non-steady state to a steady state, it is possible to obtain the value of the combined resistance R_{Combined} corresponding to the amount Δv_{Blood} of change in the blood flow v_{Blood}. Such measurement is performed for each amount Δv_{Blood} of change. The above R_{Combined} (o) is registered in the conversion table as the combined resistance R_{Combined} when the amount Δv_{Blood} of change in the blood flow v_{Blood} is o.

The laser Doppler blood flow meter 2 of the temperature measuring device constantly measures the blood flow v_{Blood} of the living body 10 at the part around the sensor 1 (step S100 in FIG. 3).

The thermal resistance derivation unit 4 of the temperature measuring device derives the combined resistance R_{Combined} by acquiring, from the conversion table of the storage unit 3, the value of combined resistance R_{Combined} corresponding to the amount Δv_{Blood} of change (=v_{Blood}−v_{Blood} (o)) of the blood flow v_{Blood} measured by the laser Doppler blood flow meter 2 (step S101 in FIG. 3).

When the blood flow v_{Blood} is within a predetermined threshold range centered on the initial value v_{Blood} (o), the thermal resistance derivation unit 4 determines that there is no change in the blood flow v_{Blood} (the amount Δv_{Blood} of change is o), and when the blood flow v_{Blood} is out of the threshold range, the thermal resistance derivation unit 4 determines that there is a change in the blood flow v_{Blood} (absolute value of the amount Δv_{Blood} of change is larger than o).

The temperature calculation unit 5 of the temperature measuring device calculates the deep body temperature T_{Core} of the living body 10 by Equation 2 above on the basis of the of result of the measurement (step S102 in FIG. 3) of the skin surface temperature Tam and the skin surface heat flux Hain by the sensor 1 and the combined resistance R_{Combined} derived by the thermal resistance deriving part 4 (step S103 in FIG. 3).

The calculation result output unit 6 of the temperature measuring device outputs the calculation result of the temperature calculation unit 5 (step S104 in FIG. 3). Examples of the output method include the display of the calculation result, the transmission of the calculation result to the outside, or the like.

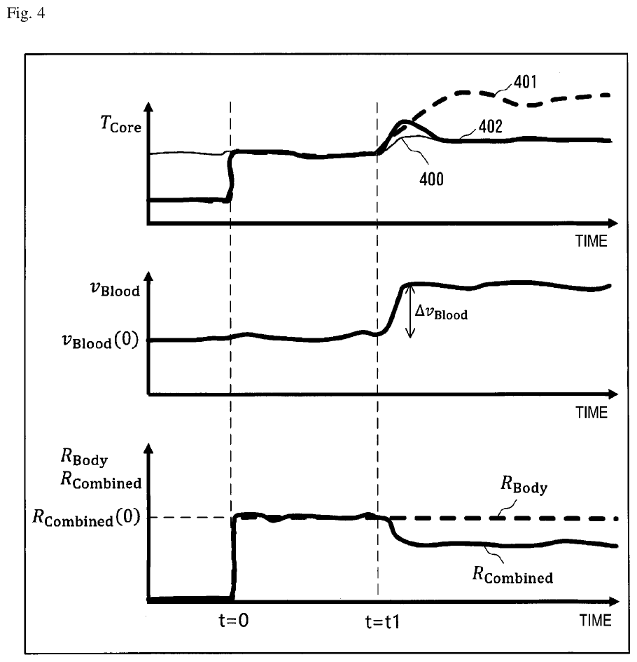

FIG. 4 is a flowchart for explaining operations of the temperature measuring device of the present embodiment. FIG. 4 illustrates an example in which the initial calibration is performed at time t=o and a person (the living body 10) wearing the sensor 1 and the sensor probe 200 starts taking a warm bath at time t=t1. In FIG. 4, 400 denotes the true value of a deep body temperature T_{Core}, 401 denotes a deep body temperature T_{Core} calculated by the method of the related art, and 402 denotes the deep body temperature T_{Core} calculated by the temperature measuring device of the present embodiment.

According to FIG. 4, it can be seen that when there is a change in the blood flow v_{Blood}, the combined resistance R_{Combined} is updated by the thermal resistance derivation unit 4 of the present embodiment from the initial value R_{Combined} (o) to a value corresponding to the amount Δv_{Blood} of change in the blood flow v_{Blood}. On the other hand, the thermal resistance R_{Body} used in the related art remains constant. Consequently, it can be seen that in the related art, an error occurs in the estimated value of the deep body temperature but in the present embodiment, the deep body temperature T_{Core} approximately equal to the true value can be estimated.

As described above, in the present embodiment, the combined resistance R_{Combined} of the living body 10 is derived on the basis of the amount Δv_{Blood} of change in the blood flow v_{Blood}, so that it is possible to reduce an error in the estimated value of the deep body temperature T_{Core} caused by a change in the blood flow v_{Blood}. In the present embodiment, the laser Doppler blood flow meter 2 is used as a blood flow meter, but other blood flow meters may be used.

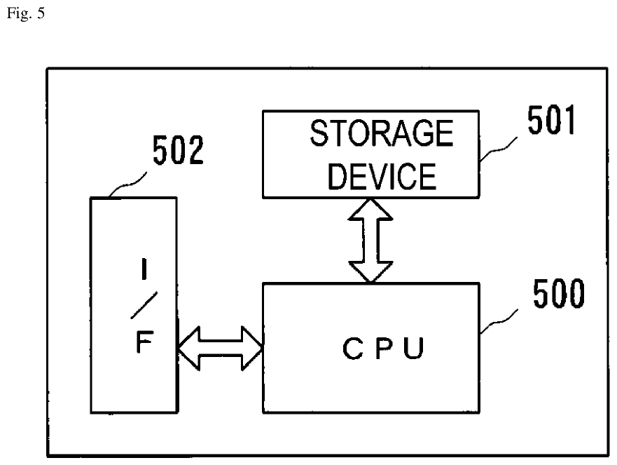

The storage unit 3, the thermal resistance derivation unit 4, the temperature calculation unit 5, and the calculation result output unit 6 described in the present embodiment can be implemented by a computer including a central processing unit (CPU), a storage apparatus, and an interface, and a program that controls these hardware resources. An example of the configuration of the computer is illustrated in FIG. 5.

The computer includes a CPU 500, a storage device 501, and an interface device (hereinafter simply referred to as I/F) 502. The sensor 1, the laser Doppler blood flow meter 2, a display device, a communication device, or the like are connected to the I/F 502. In such a computer, a program for implementing the temperature measuring method of the present invention is stored in the storage apparatus 501. The CPU 500 executes the processing described in the present embodiment in accordance with the program stored in the storage device 501.

### INDUSTRIAL APPLICABILITY

The embodiments of the present invention can be applied to a technology for measuring the internal temperature of a subject such as a living body.

### REFERENCE SIGNS LIST

1 Sensor
2 Laser Doppler blood flow meter
3 Storage unit
4 Thermal resistance derivation unit
5 Temperature calculation unit
6 Calculation result output unit
10 Living body
100 Thermal insulation member
101, 102 Temperature sensor
200 Sensor probe
201 Semiconductor laser
202 Photodiode
203 Blood flow calculation unit

1-4. (canceled)

5. A temperature measuring device comprising:
a blood flow meter configured to measure a first blood flow near a skin surface of a subject;
a sensor configured to measure a first temperature and a first heat flux of the skin surface of the subject;
a storage device configured to store a second blood flow of the subject, the second blood flow being measured before the first blood flow;
a thermal resistance deriver configured to derive a first thermal resistance of the subject based on an amount of change between the first and second blood flows; and
a temperature calculator configured to calculate an internal temperature of the subject based on the first temperature, the first heat flux, and the first thermal resistance.

6. The temperature measuring device according to claim 5, wherein the storage device stores in advance a conversion table in which a thermal resistance is registered for each amount of change in blood flow.

7. The temperature measuring device according to claim 6, wherein the thermal resistance deriver derives the first thermal resistance by acquiring, from the conversion table, a value of thermal resistance corresponding to the amount of change between the first and second blood flows.

8. The temperature measuring device according to claim 5, further comprising:
the blood flow meter configured to measure the second blood flow near the skin surface of the subject.

9. The temperature measuring device of claim 5, further comprising:
a display device to display the calculated internal temperature.

10. A temperature measuring method comprising:
measuring a first blood flow near a skin surface of a subject;
deriving a first thermal resistance of the subject based on an amount of change between the first blood flow and a second blood flow measured in advance;
measuring a first temperature and a first heat flux of the skin surface of the subject; and
calculating an internal temperature of the subject based on first temperature, the first heat flux, and the derived thermal resistance.

11. The temperature measuring method according to claim 10, further comprising:
storing in advance a calibration table in which thermal resistance corresponding to the amount of change between the first and second blood flows.

12. The temperature measuring method according to claim 10, wherein the first thermal resistance is derived by acquiring, from a conversion table stored in advance, a value of thermal resistance corresponding to the amount of change in blood flow.

13. The temperature measuring method according to claim 10, further comprising:
outputting the calculated internal temperature to a display device.

14. The temperature measuring method according to claim 10, further comprising:
sending the calculated internal temperature to a communication device.
